(19) United States
(12) Patent Application Publication
(10) Pub. No.: US 2011/0189318 A1
(43) Pub. Date: Aug. 4, 2011

(54) METHODS FOR ENHANCING THE EXPRESSION OF INTRACELLULAR REDOX-ASSOCIATED FACTORS

(76) Inventors: Misato Sugahara, Yokohama-shi (JP); Yuji Katsuta, Yokohama-shi (JP); Jotaro Nakanishi, Yokohama-shi (JP); Sam W. Lee, Newton, MA (US)

(21) Appl. No.: 12/980,051

(22) Filed: Dec. 28, 2010

Related U.S. Application Data
(60) Provisional application No. 61/299,589, filed on Jan. 29, 2010.

(51) Int. Cl. A61K 31/352; A61K 31/70; A61P 35/00; A61P 3/10; A61P 37/00; A61P 9/10; A61P 37/08; A61P 17/00; A61P 17/10; A61P 17/02; A61P 3/00; C12N 5/00; A61K 36/16; A61K 36/906

(52) U.S. Cl. 424/752; 514/456; 514/23; 435/375; 424/756

(57) ABSTRACT

The present invention provides methods for enhancing expression of a redox-associated factor, wherein kaempferol, quercetin or glycosides thereof, or any combination thereof is used as an active ingredient, and methods for enhancing expression of a redox-associated factor wherein ginkgo leaf extract, Kaempferia galanga extract or any combination thereof is used as an active ingredient.

### CROSS-REFERENCE TO RELATED APPLICATIONS

This application claims priority to U.S. Provisional Application Ser. No. 61/299,589, filed on Jan. 29, 2010, the entire contents of which are incorporated by reference herein.

### TECHNICAL FIELD

This disclosure relates to methods for enhancing expression (e.g., in vivo) of a redox-associated factor, wherein kaempferol, quercetin, glycosides thereof or any combination thereof is used as an active ingredient, and to methods for enhancing expression (e.g., in vivo) of a redox-associated factor, wherein ginkgo leaf extract, Kaempferia galanga extract or any combination thereof is used as an active ingredient.

### BACKGROUND

In organisms, redox regulations are carried out so as to maintain their existence and homeostasis. Such in vivo redox regulations are mainly based on reversible oxidation-reduction reactions on a thiol group of a cysteine residue in intracellular proteins, which may regulate various cellular functions. It has been known that a factor associated with the redox regulations (redox-associated factor) maintains the reduction condition of the environment inside a cell, and thus a redox-associated factor may play an essential role in cell viability and an important role in elimination of reactive oxygen species. If such a redox-associated factor does not function properly, endogenous reactive oxygen species are excessively produced, and thus cells can be exposed to excessive oxidative stress. Such excessive oxidative stress may oxidize biological macromolecules such as DNA, proteins and lipids, and damage them thereby causing various diseases and aging. Therefore, recently, redox-associated factors, which may have an effect of eliminating the reactive oxygen species causing oxidative stress, have been aggressively studied.

As a redox-associated factor which has an effect of preventing an organism from oxidative stress, a glutathione superfamily has been known. Glutathione is a tripeptide in mammals, and has been reported to have a reduction action resulting from intramolecular thiol groups thereof, coenzymatic function, function for generating mercapturic acid and other detoxification mechanisms, protective effect on thiol enzymes and other cellular components, function for facilitating the elimination of deleterious materials, anti-allergic effect resulting from enhanced cholinesterase activity, enzyme-activating action and the like. However, since glutathione forms a crystalline precipitate in a formulation, cells cannot directly absorb a glutathione molecule. Therefore, an ingredient for enhancing the intracellular production of glutathione can be quite useful. As such an ingredient, γ-tocopherol or δ-tocopherol has been known, and glutathione-production enhancing agents comprising such an ingredient has been disclosed (Japanese Unexamined Patent Publication (Kokai) No. 2007-284430).

In addition, as another redox-associated factor, a thioredoxin superfamily, which also has an effect of preventing an organism from oxidative stress, has been known (Saibou Kougaku, vol. 25, No. 2, 2006, p. 143-148). It has been reported that thioredoxin transgenic mice have a longer life-span (Mitsui A, et al.: Antioxid Redox Signal (2002) 4: 693-696), that thioredoxin provides resistance against cerebral infarction, diabetes or the like (Takagi Y, et al.: Proc Natl Acad Sci USA (1999) 96: 4131-36; Hotta M, et al.: J Exp Med (1998) 188: 1445-51), and that thioredoxin is effective for allergic dermatitis (Japanese Unexamined Patent Publication (Kokai) No. 2007-269671). In addition, thioredoxin has been widely used as an active oxygen eliminating agent (Japanese Unexamined Patent Publication (Kokai) No. 9-157153). However, since the thioredoxin superfamily is a macromolecule protein, even if such a compound is directly administered to a subject, it cannot be absorbed by cells. Therefore, it is desirable to enhance the in vivo expression of thioredoxin superfamily. However, as an ingredient which can enhance the expression of the family, only an Artemisia extract or a green perilla extract has been known (International publication WO 2006/033351).

As mentioned above, even if a redox-associated factor, i.e., a compound belonging to the glutathione superfamily or thioredoxin superfamily, is directly administered to a subject, it cannot be efficiently absorbed by cells, and thus a redox-regulative function or anti-oxidative function cannot be achieved in the subject. Although it has been known that the expression of a redox-associated factor is enhanced by stress, such as UV irradiation, few chemical substances acting safely and efficiently on an organism have been known, and the mechanism of in vivo expression of a redox-associated factor has not been fully elucidated. Therefore, it is still necessary to find a safe and efficient ingredient for enhancing in vivo expression of a redox-associated factor.

### SUMMARY

The problems to be solved by the present invention include, among other things, to safely and efficiently reduce oxidative stress in an organism (subject), to maintain the subject in good health and to prevent various diseases and symptoms resulting from oxidative stress by enhancing in vivo expression of a redox-associated factor.

The inventors have discovered that kaempferol and quercetin belonging to flavonols, can enhance in vivo expression and activity of a redox-associated factor, e.g., compounds belonging to the glutathione superfamily and thioredoxin superfamily. Unexpectedly, among flavonols, such a function appears specific to kaempferol and quercetin. Myricetin, which has a similar chemical structure, does not have such a function. Compared with the case of directly applying a molecule of the glutathione superfamily or the thioredoxin superfamily, applying kaempferol or quercetin can effectively enhance the expression and activity of endogenous redox-associated factors, e.g., compounds belonging to the glutathione superfamily or thioredoxin superfamily. Therefore, various endogenous oxidative stresses can be alleviated by the present invention. Flavonols including kaempferol and quercetin are innoxious and safely consumable compounds for an organism, and are widely distributed in plants such as tea leaves or the like. Therefore, endogenous excessive reactive oxygen species can be eliminated without providing any stress to an organism by using kaempferol and quercetin as an active ingredient for enhancing in vivo expression of a redox-associated factor.

Therefore, the present application comprises the following embodiments:

[1] A method for enhancing in vivo expression of a redox-associated factor, wherein kaempferol, quercetin, glycosides thereof or any combination thereof is used as an active ingredient.

[2] The method for enhancing in vivo expression of a redox-associated factor according to [1], wherein the redox-associated factor is one or more substances selected from the group consisting of thioredoxin, thioredoxin reductase, glutathione reductase, and glutaredoxin.

[3] A method for enhancing in vivo expression of a redox-associated factor, wherein ginkgo leaf extract, Kaempferia galanga extract, or any combination thereof is used as an active ingredient.

[4] The method for enhancing in vivo expression of a redox-associated factor according to [3], wherein the redox-associated factor is one or more substances selected from the group consisting of thioredoxin, thioredoxin reductase, glutathione reductase, and glutaredoxin.

Enhancing in vivo expression of a redox-associated factor can safely and efficiently reduce endogenous reactive oxygen species, and protect an organism from oxidative stress, whereby the organism can be maintained in good health, and various diseases and symptoms resulting from the oxidative stress can be avoided.

Unless defined otherwise, technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 shows enhancement for in vivo expression of a redox-associated factor by kaempferol.

FIG. 2 shows enhancement for the expression of a redox-associated factor in skin-equivalent models by kaempferol.

FIG. 3 shows a comparison of an enhancing effect by flavonoid compounds on expression of thioredoxin superfamily.

FIG. 4 shows enhancement for the expression of thioredoxin reductase by ginkgo leaf extract and Kaempferia galanga extract.

FIG. 5 shows a enhancing effect for thioredoxin reductase activity by kaempferol and quercetin.

FIG. 6 shows a suppressing effect for producing inflammatory cytokine (IL1β) by kaempferol, ginkgo leaf extract and Kaempferia galanga extract.

### DETAILED DESCRIPTION

The first embodiment of the present invention provides a method for enhancing in vivo expression of a redox-associated factor, wherein kaempferol, quercetin or glycosides thereof, or any combination thereof is used as an active ingredient.

The term “redox-associated factor” as used herein refers to a factor which oxidatively or reductively reacts with a thiol group of a cysteine residue of a protein in an organism such as a mammal, preferably a human being, and maintains the reduction condition of the environment inside the cell. In activating cells and processing signaling messengers, i.e., in signal transduction mechanism, modifications of a protein-protein interaction by oxidizing a thiol group of a cysteine residue in an intracellular protein play an important role. This oxidation/reduction (redox) of a thiol group of a cysteine residue is associated with regulations of various life phenomena, such as transcription of genes, regulations of intracellular location, synthesis and decomposition of proteins, cell proliferation and cell death. As such an endogenous redox-associated family, glutathione superfamily and thioredoxin superfamily have been known, and each of them includes a series of compounds. The redox-associated factor has been known to be involved in the elimination of reactive oxygen species, which are produced and are present in vivo.

Thioredoxin superfamily includes, in particular, thioredoxin, thioredoxin reductase and the like. Thioredoxin (TRX) was found as a coenzyme providing hydrogen to ribonucleotide reductase which is an enzyme essential for DNA synthesis in E. coli. The TRX is a protein with approximately 12 kDa, which is conserved in a variety of organisms such as prokaryotes and human beings, has a steric structure consisting of four β-strands and three α-helices, and has the active site of -Cys-Gly-Pro-Cys- (SEQ ID NO:13). The TRX has an oxidized form (S—S) which has a disulfide bond between two cysteine residues of the active site, and a reduced form (—SH, SH—) which cleaves a disulfide bond of a substrate protein and returns the oxidized form per se. The oxidized form of TRX is reduced by NADPH and thioredoxin reductase, and the reduced form is regenerated. As one of the substrate proteins, an H₂O₂ eliminating enzyme superfamily, which is generally referred to as peroxiredoxin, has been known. A reduced form of peroxiredoxin reduces H₂O₂. An oxidized peroxiredoxin functions as a substrate and is reduced by thioredoxin. An oxidized thioredoxin is reduced by thioredoxin reductase. Therefore, the thioredoxin superfamily plays an important role as a detoxifying mechanism for reactive oxygen species.

The glutathione superfamily includes, in particular, glutathione, glutathione reductase, glutaredoxin and the like. Glutathione is a tripeptide which consists of glutamic acid, cysteine, and glycine, and a large amount of glutathione is found in cells as a nonprotein thiol ingredient. Further, it has been known as a major antioxidant in hydrophilic fraction of cells. Glutathione reacts with reactive oxygen species (e.g., superoxide or hydrogen peroxide) to be a stable glutathione radical and form a dimer (GSSG: an oxidized form of glutathione), which is further regenerated as GSH (a reduced form of glutathione) after glutathione reductase transfers electrons from NADPH to GSSG. Glutaredoxin has the active site of -Cys-Pro-Tyr-Cys- (SEQ ID NO:14), which is common to thioredoxin, and thus belongs to the thioredoxin superfamily. Glutaredoxin also has an oxidized form (S—S) having a disulfide bond between the two cysteine residues of the active site and a reduced form (—SH, SH—). Similar to thioredoxin, the reduced form cleaves a disulfide bond of a substrate protein and returns the oxidized form per se. Although its biological activities are not completely understood, glutaredoxin is known to be associated with in vivo redox regulations and oxidative stress responses.

Therefore, excessive amounts of endogenous reactive oxygen species can be reduced by activating redox-associated factor(s), such as thioredoxin, thioredoxin reductase, glutathione reductase and glutaredoxin, enhancing the expression of them, and thus an organism can be protected from the oxidative stress.

As described above, the inventors recently and surprisingly discovered that kaempferol and quercetin can activate the above redox-associated factors, and enhance their expression. Flavonols including kaempferol and quercetin have the following formula.

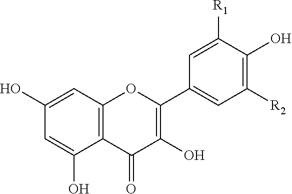

### Kaempferol: R₁═H; R₂═H

### Quercetin: R₁═OH; R₂═H

### Myricetin: R₁═OH; R₂═OH

They are flavonoid compounds which are widely distributed in plants. These flavonols are known to have an antioxidant effect, and are reported to have a blood vessel strengthening effect and anti-inflammatory effect. However, it has not been known that flavonols can enhance in vivo expression of a redox-associated factor. Further, it is surprising that kaempferol and quercetin can enhance the expression of a redox-associated factor among these flavonols, which are quite similar in chemical structure. Taking into consideration such knowledge, it is clear that kaempferol and quercetin are quite useful as an active ingredient for enhancing the expression of a redox-associated factor.

In this context, since it is well known that flavonols may form various glycosides thereof, glycosides of kaempferol or quercetin can be also used as an active ingredient in the present method for enhancing the expression of a redox-associated factor. Saccharides forming the glycoside according to the invention include, but are not limited to, aldoses, e.g., glucose, mannose, galactose, fucose, rhamnose, arabinose, and xylose, and ketoses, e.g., fructose.

As described above, since kaempferol, quercetin and glycosides thereof are ingredients widely distributed in plants, plant extracts comprising one of the ingredients are also effective for enhancing in vivo expression of a redox-associated factor. Among many plant extracts, the inventors now discovered that, in particular, ginkgo leaf extract and Kaempferia galanga extract have a remarkable effect for enhancing the expression of a redox-associated factor. Therefore, another embodiment of the invention provides a method for enhancing in vivo expression of a redox-associated factor, wherein ginkgo leaf extract, Kaempferia galanga extract, or any combination thereof is used as an active ingredient.

A blended amount of kaempferol, quercetin or glycosides thereof, or any combination thereof which is used as an active ingredient in the present method for enhancing the expression of a redox-associated factor is not limited, but is usually 0.00001 to 0.5 wt %, preferably 0.0001 to 0.001 wt %, and most preferably 0.0001 to 0.0005 wt %. Further, a blended amount of ginkgo leaf extract, Kaempferia galanga extract, or any combination thereof which is used as an active ingredient in the present method for enhancing the expression of a redox-associated factor is, for example 0.00001 to 0.5 wt %, preferably 0.0001 to 0.1 wt %, and most preferably 0.0001 to 0.01 wt % as dry weight.

In the present method for enhancing the expression of a redox-associated factor, the above active ingredients can be administered orally or parenterally as an agent for enhancing the expression of a redox-associated factor, which comprise such an active ingredient, and the agents can be prepared as, for example, an oral composition or an external composition. Further, the present method for enhancing the expression of a redox-associated factor can maintain good health, and prevent various diseases and symptoms resulting from oxidative stress. Such diseases and symptoms are, but are not limited to, cancer; diabetes; autoimmune disease such as rheumatoid arthritis, familial erythematodes, antiphospholipid antibody syndrome, dermatomyositis, familial sclerosis cutanea, Sjogren's syndrome, Guillain-Barre syndrome, chronic atrophic gastritis, aortitis syndrome, Goodpasture's syndrome, rapidly progressive glomerulonephritis, Graves' disease, chronic discoid lupus erythematosus, or recurrent fetal loss; ischemic disease such as angina pectoris, cardiac infarction, cerebral infarction, or arteriosclerosis obliterans; allergic disease such as bronchial asthma, allergic rhinitis, hay fever, allergic enterogastritis, hives, contact dermatitis, or atopic dermatitis; skin aging; pigment deposition; wrinkle; seborrhea; sunburn; burn; acne; skin sag; obesity; high blood pressure; metabolic syndrome; and anemia are exemplified. Therefore, the agent for enhancing the expression of a redox-associated factor according to the present invention can be unlimitedly used as a raw material or additive in foods, beverages, cosmetics, medicaments, and quasi-medicaments or the like.

As an oral composition, for example, common food, as well as food with health-promoting benefits, supplement, food for specified health use, modified milk for premature infants, modified milk for infants, food for infants, food for pregnant women, food for elderly people, beverage, and medicament are included. The oral composition may be prepared as a pill, powder, a granule, a lozenge, oral solution, suspension, emulsion, syrup or the like.

Moreover, the agent for enhancing the expression of a redox-associated factor, which is administered in the method according to the present invention, may comprise various carriers and additives generally used for foods/beverages, medicaments or quasi-medicaments such as anti-oxidants, in addition to the active ingredient(s). For example, the agent for enhancing the expression of a redox-associated factor may further comprise various carrier substrates, extenders, diluents, bulking agents, dispersants, excipients, coupling agent solvents (e.g., water, ethanol, plant oil), solubilizing agents, buffers, dissolution promoters, gelling agents, suspending agents, wheat flour, rice flour, starch, cornstarch, polysaccharides, milk proteins, collagen, rice oil, lecithin or the like, in addition to the active ingredient of the present invention. As additives, for example, vitamins, sweeteners, organic acids, coloring agents, fragrance, moisture preventers, fiber, electrolytes, minerals, nutrients, antioxidants, preservatives, aromatic agents, moisturizers, and natural food extracts and vegetable extracts are exemplified, but not limited to them. In addition, as antioxidants, for example, natural antioxidants such as tocopherols, flavone derivatives, phyllodulcin compounds, kojic acid, gallic acid derivatives, catechin compounds, fukiic acid, gossypol, pyrazine derivatives, sesamol, guaiaol, guaiacic acid, p-coumalic acid, nor-dihydroguaiaretic acid, sterol compounds, terpene compounds, nucleic acid base compounds, carotenoid compounds and lignan compounds; and synthetic antioxidants such ascorbic palmitate, ascorbic stearate, butylhydroxyanisole (BHA), butylhydroxytoluene (BHT), mono-t-butylhydroquinone (TBHQ) and 4-hydroxymethyl-2,6-di-t-butylphenol (HMBP) are exemplified.

As an external composition, for example, ointment, cream, protector, adhesive skin patch, pack, toner, milky lotion, lotion, bath agent, hair lotion, hair tonic, hair liquid, shampoo and rinse are included. Further, an ingredient commonly used for an external agent, for example, whitening agent, moisturizer, oily ingredient, UV absorber, surfactant, thickening agent, alcohols, powder component, coloring material, aqueous ingredient, water, and various skin nutrients can be optionally blended in the agent for enhancing the expression of a redox-associated factor used in the present method. In addition, an auxiliary agent commonly used for an external preparation, for example, a metal-chelate agent such as disodium edetate, trisodium edetate, sodium citrate, sodium polyphosphates, sodium metaphosphate, gluconic acid; caffeine, tannin, verapamil, tranexamic acid and their derivatives; a medical agent such as licorice extract, glabridin, hot water extract of Chinese quince fruit, various herbal medicines, tocopherol acetate, glycyrrhizinate, and their derivatives, or their salts; a whitening agent such as vitamin C, ascorbic acid magnesium phosphate, ascorbic acid glucoside, arbutin, kojic acid; a saccharide such as glucose, fructose, mannose, sucrose, and trehalose; vitamin A such as retinoic acid, retinol, retinol acetate, and retinol palmitate can be optionally blended in the agent for enhancing the expression of a redox-associated factor used in the present method.

Representative formulations of the agents used in the method for enhancing the expression of a redox-associated factor are shown below.

| | |
|---|---|
| | Blend ratio |
| Composition | (wt %) |

Protector A

| | |
|---|---|
| Decamethylcyclopentasiloxane | 3 |
| Methyl phenyl polysiloxane | 3 |
| Behenyl alcohol | 1 |
| 1,3-butylene glycol | 5 |
| Polyoxyethylene glyceryl isostearate | 1.5 |
| Polyoxyethylene-glycerin monostearate | 1 |
| Ginkgo leaf extract (dried residue) | 0.0001 |
| Quercetin | 0.0001 |
| Trisodium edentate | 0.1 |
| 2-ethylhexyl p-methoxycinnamate | 7 |
| Xanthan gum | 0.1 |
| Carboxy vinyl polymer | 0.3 |
| Phenoxyethanol | adequate |
| Purified water | adequate |
| Fragrance | adequate |

Protector B

| | |
|---|---|
| Petrolatum | 1 |
| Dimethylpolysiloxane | 3 |
| Methyl phenyl polysiloxane | 3 |
| Stearyl alcohol | 0.5 |
| Glycerin | 7 |
| Dipropylene glycol | 3 |
| 1,3-butylene glycol | 7 |
| Xylitol | 3 |
| Squalane | 1 |
| Isostearic acid | 0.5 |
| Stearic acid | 0.5 |
| Polyoxyethylene-glycerin monostearate | 1 |
| Glycerin monostearate | 2 |
| Potassium hydroxide | 0.05 |
| K. galanga extract (dried residue) | 0.0001 |
| Kaempferol | 0.0001 |
| Sodium acetylhyaluronate | 0.1 |
| Trisodium EDTA | 0.05 |
| 4-t-butyl-4′-methoxydibenzoylmethane | 2 |
| 2-ethylhexyl p-methoxycinnamate | 5 |
| Carboxy vinyl polymer | 0.1 |
| Phenoxyethanol | adequate |
| Purified water | adequate |
| Fragrance | adequate |

Two-layer type cream

| | |
|---|---|
| Dimethylpolysiloxane | 5 |
| Decamethylcyclopentasiloxane | 25 |
| Trimethylsiloxysilicate. | 5 |
| Copolymer of polyoxyethylene/methylpolysiloxane | 2 |
| Dipropylene glycol | 5 |
| Particulate zinc oxide (60 nm) coated with dextrin palmitate | 15 |
| Ginkgo leaf extract (dried residue) | 0.01 |
| Kaempferol | 0.0005 |
| Paraben | adequate |
| Phenoxyethanol | adequate |
| Trisodium edetate | adequate |
| 2-ethylhexyl p-methoxycinnamate | 7.5 |
| Dimethyl distearyl ammonium hectorite | 0.5 |
| Globular alkyl polyacrylate powder | 5 |
| Butyl ethyl propanediol | 0.5 |
| Purified water | adequate |
| fragrance | adequate |

Ointment

| | |
|---|---|
| Ginkgo leaf extract (dried residue) | 0.01 |
| Kaempferol | 0.001 |
| Quercetin | 0.0001 |
| Stearyl alcohol | 18.0 |
| Vegetable wax | 20.0 |
| Polyoxyethylene (20) monooleate ester | 0.25 |
| Glycerin monostearate ester | 0.3 |
| Petrolatum | 40.0 |
| Purified water | balance |

Adhesive skin patch

| | |
|---|---|
| K. galanga extract (dried residue) | 0.0001 |
| Kaempferol | 0.0001 |
| Quercetin | 0.0001 |
| Crotamiton | 3.2 |
| Panasate 875 ® | 2.5 |
| Squalane | 1.0 |
| dl-camphor | 0.07 |
| Polyoxyethylene (60E.O.)hydrogenated castor oil | 1.2 |
| Concentrated glycerin | 5.0 |
| Gelatin | 1.2 |
| Polyvinylpyrrolidone K-90 | 0.6 |
| Methylparaben | adequate |
| d-sorbitol solution | 35.0 |
| Aluminum hydroxide | 0.2 |
| Urea | 1.3 |
| Sodium sulfite | adequate |
| Sodium edetate | adequate |
| Citric acid | adequate |
| Hivis Wako 104 ® | 0.22 |
| Sodium polyacrylate | 0.24 |
| Sodium carboxymethylcellulose | 2.8 |
| Kaolin | 1.0 |
| Purified water | balance |

Iontophoresis agent

| | |
|---|---|
| Ginkgo leaf extract (dried residue) | 0.0001 |
| K. galanga extract (dried residue) | 0.0001 |
| Kaempferol | 0.0001 |
| Quercetin | 0.0001 |
| Glycerin | 5.0 |
| 1,3-butylene glycol | 5.0 |
| Hyaluronic acid | 0.05 |
| Citric acid (food) | 0.35 |
| Ion-exchange water | balance |

Candy A

| | |
|---|---|
| Ginkgo leaf extract (dried residue) | 0.5 |
| Sugar | 50.000 |
| Starch syrup | balance |
| Flavor | 1.000 |

Tablet C

| | |
|---|---|
| K. galanga extract (dried residue) | 0.1 |
| Quercetin | 0.5 |
| Sucrose esters | 4.667 |
| Methylcellulose | 2.400 |
| Glycerin | 1.667 |
| N-acetylglucosamine | 24.7998 |
| Hyaluronic acid | 20.000 |
| Vitamin E | 12.000 |
| Vitamin B6 | 1.333 |
| Vitamin B2 | 0.667 |
| α-lipoic acid | 1.333 |
| Coenzyme Q10 | 2.667 |
| Ceramide (Amorphophallus konjac extract) | 3.333 |
| L-proline | 20.000 |
| Crystalline cellulose | balance |

Soft capsule B

| | |
|---|---|
| Ginkgo leaf extract (dried residue) | 0.1 |
| Kaempferol | 0.001 |
| Quercetin | 0.0001 |
| Edible soybean oil | 35.333 |
| Royal jelly | 16.4668 |
| Maca | 2.000 |
| GABA | 2.000 |
| Bees wax | 4.000 |
| Gelatin | balance |
| Glycerin | 8.000 |
| Glycerin fatty acid esters | 7.000 |

Granule A-B

| | |
|---|---|
| Ginkgo leaf extract (dried residue) | 0.01 |
| K. galanga extract (dried residue) | 0.02 |
| Kaempferol | 0.001 |
| Soybean isoflavones | 42.2998 |
| Hydrogenated lactose | 45.000 |
| Soybean oligosaccharides | 3.000 |
| Erythritol | 3.000 |
| Dextrin | 2.500 |
| Citric acid | 2.000 |
| Flavor | balance |

Drink A

| | |
|---|---|
| Ginkgo leaf extract (dried residue) | 0.001 |
| K. galanga extract (dried residue) | 0.001 |
| Kaempferol | 0.0001 |
| Quercetin | 0.0001 |
| Hydrogenated maltose syrup | 65.3998 |
| Erythritol | 16.000 |
| Citric acid | 4.000 |
| Flavor | 2.600 |
| N-acetylglucosamine | 2.000 |
| Hyaluronic acid | 1.000 |
| Vitamin E | 0.600 |
| Vitamin B6 | 0.400 |
| Vitamin B2 | 0.200 |
| α-lipoic acid | 0.400 |
| Coenzyme Q10 | 2.400 |
| Ceramide (Amorphophallus konjac extract) | 0.800 |
| L-proline | 4.000 |
| Purified water | balance |

Cookie

| | |
|---|---|
| Ginkgo leaf extract (dried residue) | 0.5 |
| Kaempferol | 0.5 |
| Soft wheat flour | 48.7998 |
| Butter | 17.500 |
| Granulated sugar | 20.000 |
| Egg | 12.500 |
| Flavor | balance |

Granulated sugar is gradually added to a stirred butter, followed by egg, ginkgo leaf extract, K. galanga extract, kaempferol, quercetin and flavor ingredient, and further stirred. After sufficiently blending the mixture, homogeneously-dispersed soft wheat flour is added, the mixture is gently stirred, and the blended dough is placed in a refrigerator. Subsequently, the cooled dough is shaped, and then baked at 170° C. for 15 minutes to make cookies.

### EXAMPLES

### Example 1

### Identification of Agents Enhancing the Expression of Thioredoxin Superfamily

Normal human epidermal keratinocytes (KK-4009, KURABO) were cultured in Defined Keratinocyte-SFM medium (10744-019, GIBCO) with 5% CO₂ under high humidity at 37° C. For the cells with 100% of final density, the medium was replaced with EpiLife™ Calcium-Free Phenol Red Free medium (M-EPICF/PRF-500, KURABO) whose calcium concentration had been adjusted to 1.5 mM. Twenty-four hours later, kaempferol (K0018, Tokyo Chemical Industry Co., Ltd), vitamin E (95240, FULKA), or lipoic acid ((+/−)-α-lipoic acid (T1395, SIGMA) was added so as to have a final concentration of 10 μM.

Twenty-four hours after adding the above agents, RNA was isolated by using QIAZOL Lysis Reagent (79306, QIAGEN) and purified by using RNeasy Kit (74106, QIAGEN). Subsequently, mRNAs of thioredoxin reductase, thioredoxin, glutathione reductase, glutaredoxin, and the ribosomal protein as an internal standard were quantified by the LightCycler™ software (Roche) using QuantiTect™ SYBR Green RT-PCR Kit (204243, QIAGEN). The Sequences of the used primers are shown below.

Thioredoxin reductase/Forward primer:

| | |
|---|---|
| TGCTGGCAATAGGAAGAGATGGCTTGCAC | (SEQ ID NO: 1) |

Thioredoxin reductase/Reverse primer:

| | |
|---|---|
| GCAATCTTCCTGCCTGCCTGGATTGCAACTGG | (SEQ ID NO: 2) |

Thioredoxin/Forward primer:

| | |
|---|---|
| TCGGTCCTTACAGCCGCTCGTCAGACTCCA | (SEQ ID NO: 3) |

Thioredoxin/Reverse primer:

| | |
|---|---|
| AGGCCCACACCACGTGGCTGAGAAGTCAAC | (SEQ ID NO: 4) |

Glutathione reductase/Forward primer:

| | |
|---|---|
| GATCCTGTCAGCCCTGGGTTCTAAGACATC | (SEQ ID NO: 5) |

Glutathione reductase/Reverse primer:

| | |
|---|---|
| TAACCATGCTGACTTCCAAGCCCGACAA | (SEQ ID NO: 6) |

Glutaredoxin/Forward primer:

| | |
|---|---|
| ATCACAGCCACCAACCACACTAACGAGA | (SEQ ID NO: 7) |

Glutaredoxin/Reverse primer:

| | |
|---|---|
| GTTACTGCAGAGCTCCAATCTGCTTTAGCC | (SEQ ID NO: 8) |

A ribosomal protein/Forward primer:

| | |
|---|---|
| ACAGAGGAAACTCTGCATTCTCGCTTCCTG | (SEQ ID NO: 9) |

A ribosomal protein/Reverse primer:

| | |
|---|---|
| CACAGACAAGGCCAGGACTCGTTTGTACC | (SEQ ID NO: 10) |

The mRNA amounts of thioredoxin reductase, thioredoxin, glutathione reductase and glutaredoxin were divided by the mRNA amount of the ribosomal protein to obtain the mRNA expression levels.

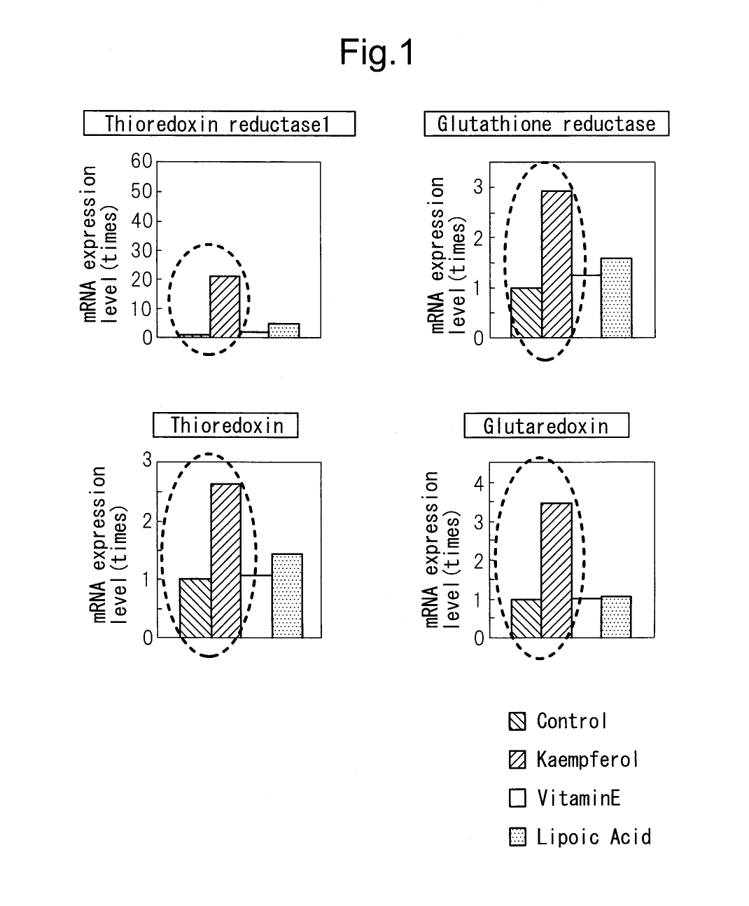

It was found that kaempferol significantly enhanced the expression of the redox-associated factors, as compared with vitamin E and lipoic acid which are known to have an antioxidant effect (FIG. 1).

### Example 2

### Effect of Kaempferol in Enhancing the Expression of Redox-Associated Factors in Skin-Equivalent Model

Skin-equivalent models were made from normal human keratinocytes (Invitrogen), normal human fibroblasts (ATCC) and collagen I (Sigma), and cultured in Dulbecco's Modified Eagle's Medium (D-MEM) (Invitrogen) with 5% CO₂ under high humidity at 37° C. Two μM or 10 μM kaempferol was added into the culture medium, and the models were cultured for twenty-four hours. Then RNA was isolated from epidermal layer of the skin-equivalent models by using a QIAGEN mRNA isolation kit. Subsequently, mRNAs of thioredoxin reductase and glutathione reductase, and 36B4 as an internal standard, were quantified by q-PCR (quantitative PCR by Bio-Rad MyiQ™ single color real time PCR detection system) using the Real-time PCR kit (Bio-Rad). The primers were purchased from QIAGEN (TXNRD2: #QT00070371; GSR; #QT00038325). The amounts of thioredoxin reductase and glutathione reductase were divided by the mRNA amount of 36B4 to obtain the mRNA expression level.

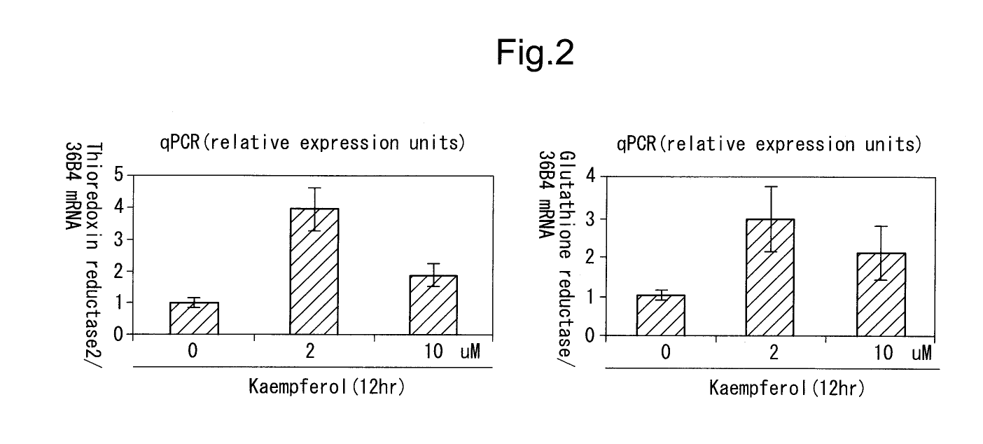

Kaempferol significantly enhanced the expression of the redox-associated factors in skin-equivalent models (FIG. 2).

### Example 3

### Screen for Flavonoid Compounds Enhancing Expression of Thioredoxin Superfamily

Normal human epidermal keratinocytes (KK-4009, KURABO) were cultured in Defined Keratinocyte-SFM medium (10744-019, GIBCO) with 5% CO₂ under high humidity at 37° C. For the cells with 100% of final density, the medium was replaced with EpiLife™ Calcium-Free Phenol Red Free medium (M-EPICF/PRF-500, KURABO) whose calcium concentration had been adjusted to 1.5 mM. Twenty-four hours later, each of the following agents was added so as to have a final concentration of 10 μM.

### Kaempferol (K0018, Tokyo Chemical Industry Co., Ltd)

### Quercetin (173-00403, Wako Pure Chemical Industries, Ltd.)

### Myricetin (70050, FLUKA)

Epigallocatechin gallate (E4143, CIAL)

### Hesperidin (086-07342, Wako Pure Chemical Industries, Ltd.)

### Apigenin (012-18913, Wako Pure Chemical Industries, Ltd.)

### Genistein (070-04681, Wako Pure Chemical Industries, Ltd.)

Twenty-four hours after adding the above agents, the mRNA expression levels of the redox-associated factors were quantified by the same method as Example 1.

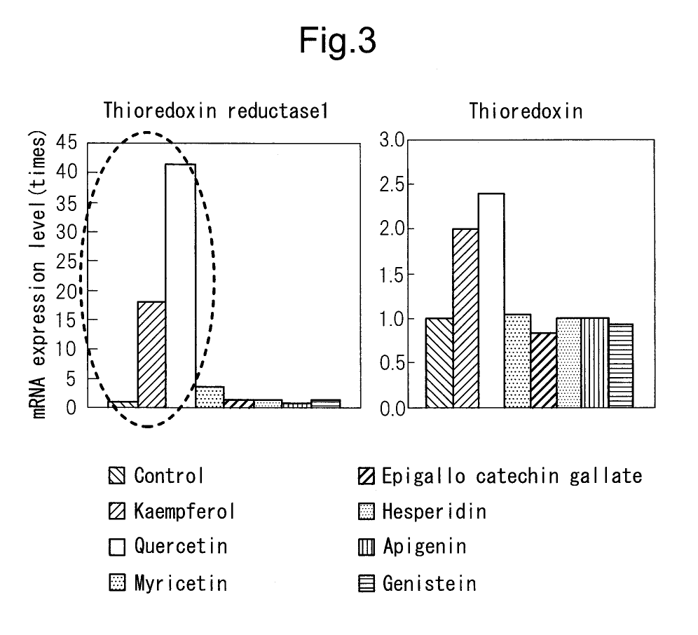

Among the above flavonoid compounds, kaempferol and quercetin were found to significantly enhance the expression of the thioredoxin superfamily (FIG. 3). The remaining compounds did not significantly enhance expression (FIG. 3).

### Example 4

### Identification of Plant Extracts Enhancing the Expression of Thioredoxin Superfamily

Normal human epidermal keratinocytes (KK-4009, KURABO) were cultured in Defined Keratinocyte-SFM medium (10744-019, GIBCO) with 5% CO₂ under high humidity at 37° C. For the cells with 100% of final density, the medium was replaced with EpiLife™ Calcium-Free Phenol Red Free medium (M-EPICF/PRF-500, KURABO) whose calcium concentration had been adjusted to 1.5 mM. Twenty-four hours later, each of the following agents was added so as to have a final concentration of 0.1%.

K. galanga extract: which is obtained by the extraction of Kaempferia galanga rhizomes with 50% 1,3-butylene glycol;
Ginkgo leaf extract: which is obtained by resolving 50% ethanol extract from Ginkgo biloba L. leaves in 1,3-butylene glycol;
Rubus suavissimus extract: which is obtained by resolving boiling-water extract from Rubus suavissimus Shugan Lee. (Rosaseae) in 50% 1,3-butylene glycol.

Twenty-four hours after adding the above agents, the mRNA expression levels of the redox-associated factors were quantified by the same method as Example 1.

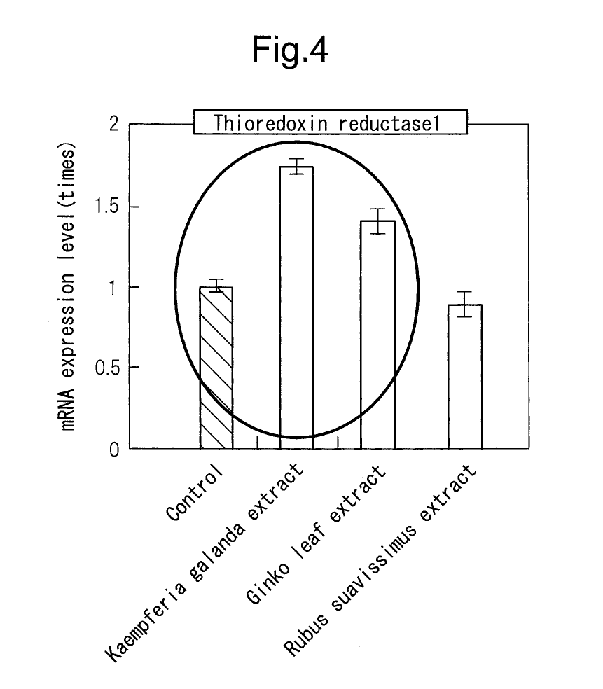

As a result, among the plant extracts containing kaempferol, quercetin and/or glycosides thereof, it was verified that ginkgo leaf extract and K. galanga extract particularly enhanced the expression of the thioredoxin superfamily relative to control (FIG. 4).

### Example 5

### Thioredoxin Reductase Activity-Enhancing Effect

Normal human epidermal keratinocytes (KK-4009, KURABO) were cultured in Defined Keratinocyte-SFM medium (10744-019, GIBCO) with 5% CO₂ under high humidity at 37° C. For the cells with 100% of final density, the medium was replaced with EpiLife™ Calcium-Free Phenol Red Free medium (M-EPICF/PRF-500, KURABO) whose calcium concentration had been adjusted to 1.5 mM. Twenty-four hours later, kaempferol (K0018, Tokyo Chemical Industry Co., Ltd) or quercetin (173-00403, Wako Pure Chemical Industries, Ltd.) was added so as to have a final concentration of 10 μM. Twenty-four hours after adding the agents, the cells were dissolved in lysis buffer, and the insoluble fraction was removed by centrifugation at 14,000 rpm, 4° C. for 15 minutes. The amount of total protein in the soluble fraction was quantified, and then thioredoxin reductase activity was measured by the method of Arne Holmgren et al. (METHODS IN ENZYMOLOGY, 1995, 252, 199-208).

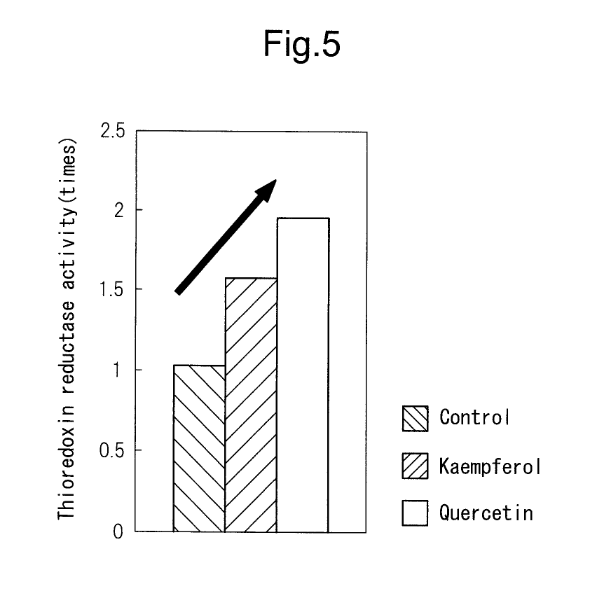

Kaempferol and quercetin were found to enhance thioredoxin reductase activity relative to control (FIG. 5).

### Example 6

### Suppression Effects of Kaempferol, K. galanga Extract and Ginkgo Leaf Extract on the Expression of an Inflammatory Cytokine (IL1β)

Normal human epidermal keratinocytes (KK-4009, KURABO) were cultured in Defined Keratinocyte-SFM medium (10744-019, GIBCO) with 5% CO₂ under high humidity at 37° C. For the cells with 100% of final density, the medium was replaced with EpiLife™ Calcium-Free Phenol Red Free medium (M-EPICF/PRF-500, KURABO) in which calcium concentration had been adjusted to 1.5 mM. Twenty-four hours later, kaempferol was added to have 10 μM of final concentration and each of the following plant extracts was added to have a final concentration of 0.1%.

kaempferol (K0018, Tokyo Chemical Industry Co., Ltd);
K. galanga extract: which is obtained by the extraction of Kaempferia galanga rhizomes with 50% 1,3-butylene glycol;
Ginkgo leaf extract: which is obtained by resolving 50% ethanol extract from Ginkgo biloba L. leaves in 1,3-butylene glycol.

Twenty-four hours after adding the above agents, RNA was isolated by the same method as Example 1, and the mRNA expression level of the inflammation-associated factor (IL1β) was quantified. The used primers are shown below.

IL1β/Forward primer

| | |
|---|---|
| GGCCATGGACAAGCTGAGGAAGATGCTG | (SEQ ID NO: 11) |

IL1β/Reverse primer

| | |
|---|---|
| TGCATCGTGCACATAAGCCTCGTTATCCC | (SEQ ID NO: 12) |

The mRNA amount of IL1β was divided by the mRNA amount of the ribosomal protein to obtain the mRNA expression level.

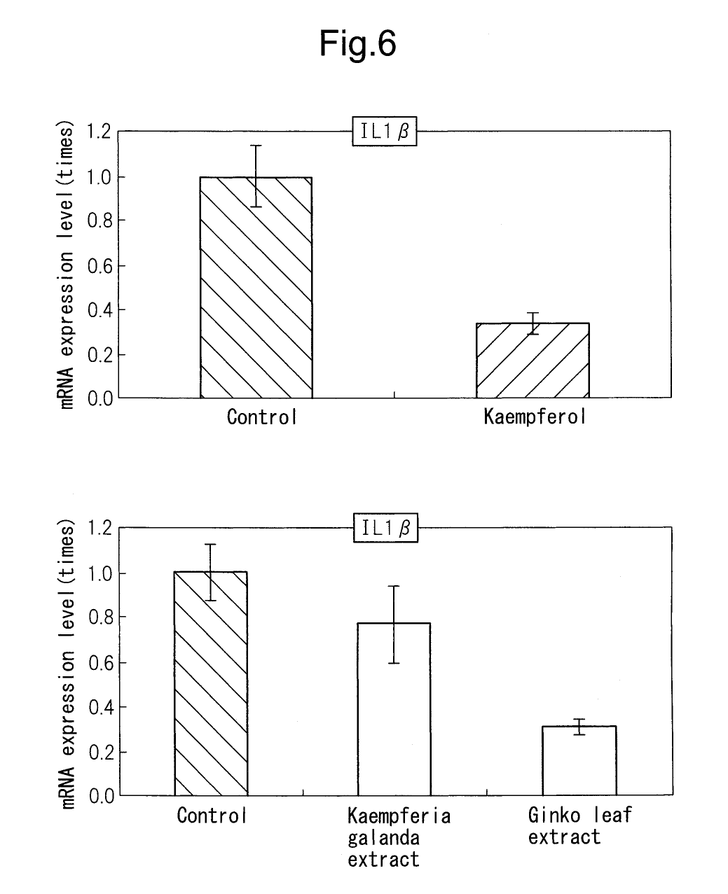

Kaempferol, K. galanga extract and ginkgo leaf extract were found to have an anti-inflammatory effect including suppression of the inflammatory cytokine IL1β (FIG. 6).

### Other Embodiments

A number of embodiments of the invention have been described. Nevertheless, it will be understood that various modifications may be made without departing from the spirit and scope of the invention. Accordingly, other embodiments are within the scope of the following claims.

We claim:

1. A method for enhancing in vivo expression of a redox-associated factor, wherein kaempferol, quercetin or glycosides thereof, or combination thereof is used as an active ingredient.

2. The method for enhancing the expression of an endogenous redox-associated factor according to claim 1, wherein the redox-associated factor is one or more substances selected from the group consisting of thioredoxin, thioredoxin reductase, glutathione reductase and glutaredoxin.

3. A method for enhancing in vivo expression of a redox-associated factor, wherein ginkgo leaf extract, Kaempferia galanga extract or a combination thereof is used as an active ingredient.

4. The method for enhancing the expression of an endogenous redox-associated factor according to claim 3, wherein the redox-associated factor is one or more substances selected from the group consisting of thioredoxin, thioredoxin reductase, glutathione reductase and glutaredoxin.

5. A method of treating or preventing a disease, disorder, or condition or alleviating one or more symptoms of a disease, disorder or condition, the method comprising
identifying a patient suffering from or at risk for the disorder; and
administering to the patient a composition comprising kaempferol, quercetin, or glycosides thereof, or a combination thereof,
wherein the disorder is cancer; diabetes; autoimmune disease such as rheumatoid arthritis, familial erythematodes, antiphospholipid antibody syndrome, dermatomyositis, familial sclerosis cutanea, Sjogren's syndrome, Guillain-Barre syndrome, chronic atrophic gastritis, aortitis syndrome, Goodpasture's syndrome, rapidly progressive glomerulonephritis, Graves' disease, chronic discoid lupus erythematosus, or recurrent fetal loss; ischemic disease such as angina pectoris, cardiac infarction, cerebral infarction, or arteriosclerosis obliterans; allergic disease such as bronchial asthma, allergic rhinitis, hay fever, allergic enterogastritis, hives, contact dermatitis, or atopic dermatitis; skin aging; pigment deposition; wrinkle; seborrhea; sunburn; burn; acne; skin sag; obesity; high blood pressure; metabolic syndrome; or anemia.

6. A method of treating or preventing a disease, disorder, or condition or alleviating one or more symptoms of a disease, disorder or condition, the method comprising
identifying a patient suffering from or at risk for the disorder; and
administering to the patient a composition comprising ginkgo leaf extract or Kaempferia galanga extract, or a combination thereof,
wherein the disorder is cancer; diabetes; autoimmune disease such as rheumatoid arthritis, familial erythematodes, antiphospholipid antibody syndrome, dermatomyositis, familial sclerosis cutanea, Sjogren's syndrome, Guillain-Barre syndrome, chronic atrophic gastritis, aortitis syndrome, Goodpasture's syndrome, rapidly progressive glomerulonephritis, Graves' disease, chronic discoid lupus erythematosus, or recurrent fetal loss; ischemic disease such as angina pectoris, cardiac infarction, cerebral infarction, or arteriosclerosis obliterans; allergic disease such as bronchial asthma, allergic rhinitis, hay fever, allergic enterogastritis, hives, contact dermatitis, or atopic dermatitis; skin aging; pigment deposition; wrinkle; seborrhea; sunburn; burn; acne; skin sag; obesity; high blood pressure; metabolic syndrome; or anemia.

7. A method of enhancing the expression of a redox-associated factor in a cell, the method comprising contacting the cell with a composition comprising kaempferol, quercetin, glycosides thereof, or a combination thereof, thereby enhancing expression of a redox-associated factor in the cell.

8. The method according to claim 7, wherein the redox-associated factor is selected from the group consisting of thioredoxin, thioredoxin reductase, glutathione reductase and glutaredoxin.

9. The method of claim 7, further comprising identifying a cell in need of enhanced expression of a redox-associated factor in the cell.

10. The method of claim 7, wherein the cell is in a subject.

11. The method of claim 10, further comprising identifying an organism in need of enhanced expression of a redox-associated factor in a cell in the subject.

12. The method of claim 7, wherein the composition comprises kaempferol or a glycoside thereof.

13. The method of claim 7, wherein the composition comprises quercetin or a glycoside thereof.

14. A method of enhancing the expression of a redox-associated factor in a cell, the method comprising contacting the cell with a composition comprising ginkgo leaf extract, Kaempferia galanga extract or a combination thereof, thereby enhancing expression of a redox-associated factor in the cell.

15. The method according to claim 14, wherein the redox-associated factor is selected from the group consisting of thioredoxin, thioredoxin reductase, glutathione reductase and glutaredoxin.

16. The method of claim 14, further comprising identifying a cell in need of enhanced expression of a redox-associated factor in the cell.

17. The method of claim 14, wherein the cell is in a subject.

18. The method of claim 15, further comprising identifying an organism in need of enhanced expression of a redox-associated factor in a cell in the subject.

19. The method of claim 14, wherein the composition comprises ginkgo leaf extract.

20. The method of claim 14, wherein the composition comprises Kaempferia galanga extract.


SEQUENCE LISTING (14 sequences)

SEQ ID NO: 1 - DNA, 29 bp, Artificial
  forward primer
tgctggcaataggaagagatggcttgcac

SEQ ID NO: 2 - DNA, 32 bp, Artificial
  reverse primer
gcaatcttcctgcctgcctggattgcaactgg

SEQ ID NO: 3 - DNA, 30 bp, Artificial
  forward primer
tcggtccttacagccgctcgtcagactcca

SEQ ID NO: 4 - DNA, 30 bp, Artificial
  reverse primer
aggcccacaccacgtggctgagaagtcaac

SEQ ID NO: 5 - DNA, 30 bp, Artificial
  forward primer
gatcctgtcagccctgggttctaagacatc

SEQ ID NO: 6 - DNA, 28 bp, Artificial
  reverse primer
taaccatgctgacttccaagcccgacaa

SEQ ID NO: 7 - DNA, 28 bp, Artificial
  forward primer
atcacagccaccaaccacactaacgaga

SEQ ID NO: 8 - DNA, 30 bp, Artificial
  reverse primer
gttactgcagagctccaatctgctttagcc

SEQ ID NO: 9 - DNA, 30 bp, Artificial
  forward primer
acagaggaaactctgcattctcgcttcctg

SEQ ID NO: 10 - DNA, 29 bp, Artificial
  reverse primer
cacagacaaggccaggactcgtttgtacc

SEQ ID NO: 11 - DNA, 28 bp, Artificial
  forward primer
ggccatggacaagctgaggaagatgctg

SEQ ID NO: 12 - DNA, 29 bp, Artificial
  reverse primer
tgcatcgtgcacataagcctcgttatccc

SEQ ID NO: 13 - protein, 4 aa, Artificial
  active site
CGPC

SEQ ID NO: 14 - protein, 4 aa, Artificial
  active site
CPYC